(19) United States
(12) Patent Application Publication
(10) Pub. No.: US 2023/0293767 A1
(43) Pub. Date: Sep. 21, 2023

(54) FILLING/CUSHIONING MEMBER FOR SKIN GRAFTS ON AURICULAR SKIN DEFECTS

(71) Applicants: National University Corporation University of Toyama, Toyama (JP); SAKURA SEIKI CO., LTD., Nagano (JP)

(72) Inventors: Michiro FUJISAKA, Toyama (JP); Hideo SHOJAKU, Toyama (JP); Shinsuke ITO, Toyama (JP); Toshiko YOSHIDA, Toyama (JP); Motonori OKABE, Toyama (JP); Chika SOKO, Toyama (JP); Masahiko ARAKAWA, Nagano (JP)

(21) Appl. No.: 18/021,113

(22) PCT Filed: Oct. 30, 2020
(86) PCT No.: PCT/JP2020/040753

§ 371 Date: Feb. 13, 2023

(51) Int. Cl. A61L 27/36; A61L 27/54; A61L 27/60

(52) U.S. Cl. CPC A61L 27/3604; A61L 27/3691; A61L 27/54; A61L 27/60; A61L 2430/14

(57) ABSTRACT

The present invention addresses the problem of providing a material as a scaffold used to prevent poor skin grafting when there is a defect in the perichondrium or periosteum or a defect in subcutaneous tissue. As a solution, there is provided a dry amniotic membrane manufactured according to a specific drying process. In more detail, a raw amniotic membrane placed in a processing tank (10) is continuously heated by a far-infrared heater (14) provided inside the processing tank (10), while performing a decompression operation where the interior of the processing tank (10) is placed in a decompressed state and irradiation of the raw amniotic membrane with microwaves from a microwave generating device (30) provided inside the processing tank (10) to apply energy to water molecules present inside the amniotic membrane and cause drying during a pressure recovery operation that slightly raises the pressure inside the depressurized processing tank (10) toward atmospheric pressure. By providing amniotic membrane, which has been dried by repeating the above process a plurality of times to retain its cellular and tissue structure, as a scaffold used to prevent poor skin grafting when there is a defect in the perichondrium or periosteum or a defect in the subcutaneous tissue, it is possible to enhance the healing effect of a skin graft.

### TECHNICAL FIELD

The present invention relates to a filling/cushioning member used for skin grafts on auricular skin defects.

### BACKGROUND ART

It is known that when skin grafting is performed on a defect following skin removal treatment due to the onset of cancer or the like, there are parts of the body where engraftment is difficult. For auricular cancer (basal cell carcinoma), even when skin grafting is performed on an auricular skin defect, infection is likely and engraftment failure is common.

Amniotic membrane has come into use as an effective dressing for intractable wounds.

### CITATION LIST

### Patent Literature

Patent Literature 1: Japanese Laid-open Patent Publication No. 2007-54015 Non Patent Literature
Non Patent Literature 1: Gruss, J. S. & Jirsch, D. W. Human amniotic membrane: A versatile wound dressing. Can. Med. Assoc. J. 118, 1237-1246 (1978).
Non Patent Literature 2: Okabe, M. et al. Hyperdry human amniotic membrane is useful material for tissue engineering: Physical, morphological properties, and safety as the new biological material. J. Biomed. Mater. Res.-Part A 102, 862-870 (2014).

### SUMMARY OF INVENTION

### Technical Problem

Amniotic membrane is a tough biological membrane composed of collagen and elastic fibers, and raw amniotic membrane has been reported as being a useful covering material for trauma and burns (see Non Patent Literature 1).

However, raw amniotic membrane has not been available immediately when needed and has been complicated to store and handle, which has limited its use in actual clinical practice. On the other hand, dry amniotic membrane (or “hyper-dry amniotic membrane”: hereinafter, “HD-AM”) produced by a specific drying process has also been announced (see Patent Literature 1 and Non Patent Literature 2).

Although skin grafting is performed on defects produced by treatment that removes auricular skin at the onset of cancer or the like, engraftment can be difficult depending on the location. For auricular cancer (basal cell carcinoma) in particular, even when skin grafting is performed on an auricular skin defect, infection is likely and engraftment failure is common.

Negative pressure wound therapy (NPWT) on defects to the perichondrium or subcutaneous tissue is a wound care method in which a foam material, such as polyurethane, is placed on a wound, the entire wound is covered with a film material, and negative pressure is continuously applied to the covered wound to promote wound healing.

As an advantage of the present method, the research conducted here found that a favorable scaffold could be formed by placing hyper-dry amniotic membrane on cartilage that has become exposed due to a defect in the perichondrium and/or subcutaneous tissue.

Conventional skin grafting was poor and has not had a material with an effect of directly promoting healing. The sole benefit was avoiding necrosis of the affected area.

### Solution to Problem

The present inventors established that HD-AM functions as a scaffold for cells and promotes tissue regeneration, which contributes to engraftment after a skin graft, thereby completing the present invention.

A filling/cushioning member according to an aspect of the present invention is a restorative member used for skin grafting on auricular skin defects, composed of a dry amniotic membrane produced by performing a drying process on raw amniotic membrane that surrounds a fetus of an animal, including a human, wherein the dry amniotic membrane has been dehydrated and dried so as to enable storage in an aseptic dry atmosphere, and amniotic membrane produced by rehydrating the dry amniotic membrane by immersion in water or a buffer retains epithelial cells, basement membrane, and connective tissue constituting the raw amniotic membrane.

By using this configuration, the active effect of promoting wound healing promotes engraftment of a skin graft.

The filling/cushioning member according to the present invention may be a filling/buffering member used to promote engraftment of a skin graft.

### Advantageous Effects of Invention

By using the filling/cushioning member according to the present invention during a skin grafting operation on a perichondrium defect or subcutaneous tissue defect, it is possible to promote engraftment of a skin graft through the active effect of amniotic membrane in promoting wound healing.

### BRIEF DESCRIPTION OF DRAWINGS

FIG. 1 is a diagram useful in explaining a first skin graft following excision of auricular cancer (basal cell carcinoma). Excision was performed, and skin grafting was performed on the resulting auricular skin defect. Pathological results from a month and a half later found residual cancer, exposed cartilage, infected necrotic areas, pus discharge, and no engraftment of the skin graft.

FIG. 2 is a diagram explaining excision of recurrent basal cell carcinoma. 1. With a safety margin, the tissue was collected around the circumference and submitted for rapid pathological diagnosis. The absence of cancer cells was confirmed. 2. While observing with an endo scope, a circumferential incision was made in the skin of the external auditory canal at a position just before the eardrum. A resection stump was created on the external auditory canal side.

FIG. 3 is a diagram explaining a skin graft to which HD-AM has been applied. In FIG. 3A, the HD-AM was applied over the external auditory canal and the auricular cartilage. In FIG. 3B, a skin graft was performed on top of the HD-AM.

FIG. 4 is a diagram useful in explaining treatment results of skin grafting with HD-AM. Two months later, the skin graft had taken root and was in good condition.

FIG. 5 depicts drying equipment for preparing HD-AM.

### DESCRIPTION OF EMBODIMENTS

### Overview of Embodiments

A dried amniotic membrane manufactured by a specified drying process (called “hyperdrying”) is, for example, the dried amniotic membrane described in Patent Literature 1. That is, raw amniotic membrane placed in a processing tank is continuously heated by a far-infrared heater provided inside the processing tank, and a depressurization operation, in which the inside of the processing tank is placed in a depressurized state, and irradiation of the raw amniotic membrane with microwaves from a microwave generating device provided inside the processing tank to apply energy to water molecules present inside the amniotic membrane and cause drying during a pressure recovery operation that slightly raises the pressure inside the depressurized processing tank toward atmospheric pressure were performed. In dried amniotic membrane (HD-AM) manufactured by repeating the above process a plurality of times, the amniotic membrane cells themselves are inactivated but the cell and tissue structures are retained.

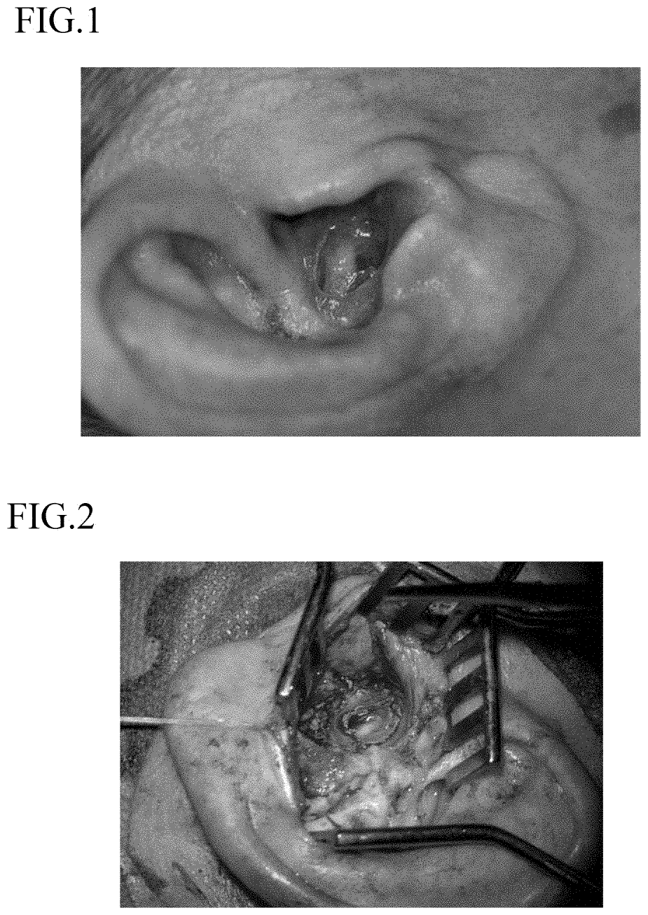

Excision of auricular cancer (basal cell carcinoma) was performed at another department, and then skin grafting was performed on the auricular skin defect. However, residual cancer was found at the wound, partial infection occurred, and engraftment failed (see FIG. 1).

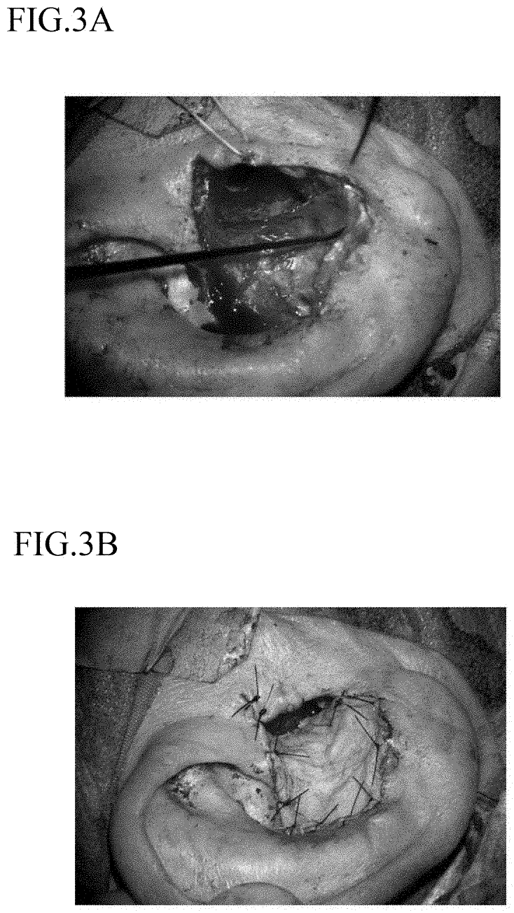

A safety margin was provided from the previously excised site, and after confirming through rapid pathological diagnosis that no cancer remained, the cancer was removed as a mass (see FIG. 2). HD-AM was applied to the surface of the exposed auricular cartilage (see FIG. 3A), and then harvested skin was grafted on top (see FIG. 3B).

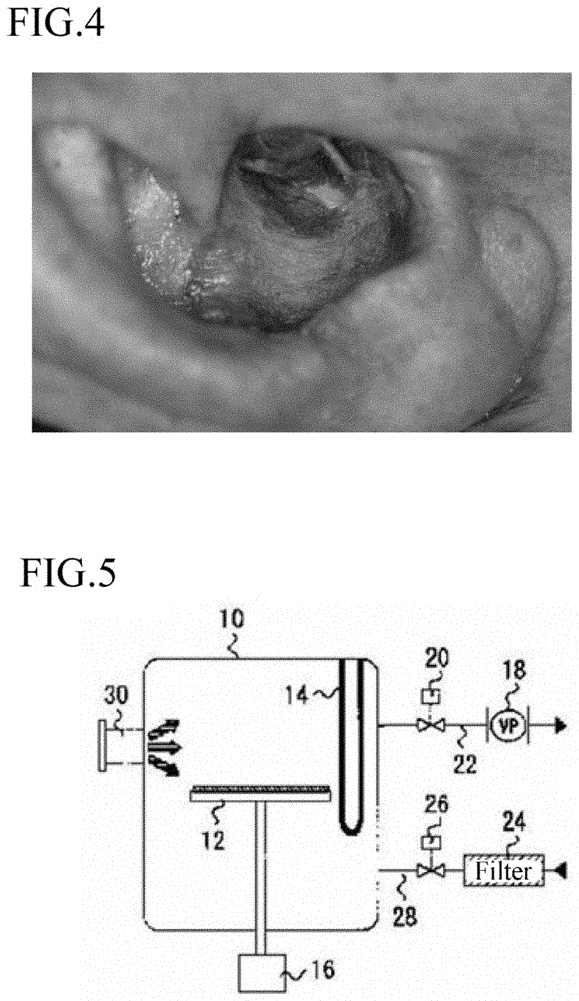

Two months later, the postoperative course was favorable (FIG. 4).

In the present embodiment, HD-AM is placed as a filling/cushioning member on a perichondrium defect or a subcutaneous tissue defect. By placing the HD-AM as a scaffold between a skin graft and a perichondrium defect or a subcutaneous tissue defect, the active effects of the amniotic membrane, such as the promotion of wound healing and formation of a scaffold, are promoted.

### Manufacture of HD-AM

Using the drying device depicted in FIG. 5, a raw amniotic membrane was dried with vacuum, far-infrared, and microwave devices set at the conditions given below.

The drying device in FIG. 5 is equipped, inside a processing tank 10, with a rotary table 12 on which raw amniotic membrane is placed, and a far-infrared heater 14 as a heating means. Also, to reduce the pressure inside the processing tank 10, a solenoid valve 20 and a vacuum pump 18 are connected to the processing tank 10. Also, to repressurize the processing tank 10 interior, a solenoid valve 26 and a filter 24 are connected to the processing tank 10.

The processing tank 10 is also provided with a microwave irradiation device 30 which performs, when a pressure recovery operation has been performed from a reduced pressure state, drying while applying energy to water molecules in the amniotic membrane.

Drying tank heating temperature: 50° C., FIR: 50° C., Stop valve: 37%, Maximum ultimate pressure 0.34 kPa, Dry running maximum ultimate pressure 0.33 kPa

Drying Method
(1) Depressurization 180 sec
(2) Pressure recovery 30 sec (with stop valve 37% open)
Commence emission of microwaves
0.1 kw for 180 sec (pressure recovery continues)
(3) Depressurization 180 sec
(4) Repeat (2) and (3) thereafter
(5) End drying manually by checking the ultimate pressure after 180 sec of depressurization in (3) is (0.30 to 0.35 kPa).
   Process complete when atmospheric pressure is restored

### INDUSTRIAL APPLICABILITY

Dried amniotic membrane (HD-AM) is manufactured by a specific drying process, that is, raw amniotic membrane that has been placed inside a processing tank is continuously heated by a far-infrared heater provided inside the processing tank while performing a depressurization operation that reduces the pressure inside the processing tank and irradiation of the raw amniotic membrane with microwaves from a microwave generating device provided inside the processing tank to apply energy to the water molecules present inside the amniotic membrane and dry the amniotic membrane during a pressure recovery operation that slightly raises the pressure inside the depressurized processing tank toward atmospheric pressure. By repeating the above multiple times, it is possible to improve storage stability and handling of the dried amniotic membrane while preserving the cell and tissue structures. By using this HD-AM as a scaffold to prevent poor skin grafting on perichondrium/periosteal defects and subcutaneous tissue defects, it is possible to enhance the healing effect of skin grafts.

What is claimed is:

1. A filling/cushioning member for perichondrium and subcutaneous tissue that is a restorative member used for skin grafting on auricular skin defects,
the filling/cushioning member comprising a dry amniotic membrane produced by performing a drying process on raw amniotic membrane that surrounds a fetus of an animal, including a human,
wherein the dry amniotic membrane has been dehydrated and dried so as to enable storage in an aseptic dry atmosphere, and
amniotic membrane produced by rehydrating the dry amniotic membrane by immersion in water or a buffer retains epithelial cells, basement membrane, and connective tissue constituting the raw amniotic membrane.

2. The filling/cushioning member according to claim 1 used for a skin graft on an auricular skin defect.
